(19) United States
(12) Patent Application Publication
(10) Pub. No.: US 2013/0180258 A1
(43) Pub. Date: Jul. 18, 2013

(54) PROCESS FOR PRODUCING AN OXYGEN-CONTAINING COMPOUND

(71) Applicant: Luis Angel Robels Macias, Paris (FR)

(72) Inventor: Luis Angel Robels Macias, Paris (FR)

(73) Assignee: TOTAL RAFFINAGE CHIMIE, Courbevoie (FR)

(21) Appl. No.: 13/877,019

(22) PCT Filed: Sep. 26, 2011
(86) PCT No.: PCT/FR11/52222

§ 371 Date: Mar. 29, 2013

(30) Foreign Application Priority Data
Sep. 29, 2010 (FR) 10 57872

(51) Int. Cl. C12P 7/54; C12P 7/16; C12P 7/52; C12P 7/06

(52) U.S. Cl. CPC C12P 7/54; C12P 7/06; C12P 7/16; C12P 7/52
USPC 60/772; 435/161; 435/160; 435/140; 435/141

(57) ABSTRACT

The subject of the invention is a process for producing an oxygen-containing compound chosen from C₁ to C₄ alcohols or carboxylic acids, and mixtures thereof, comprising the following steps:
a step of catalytic decomposition of a hydrocarbon-based feedstock to give carbon, said step also producing hydrogen; the
partial oxidation of the resulting carbon to give CO, using CO₂, the CO stream being separated from the hydrogen stream; and the
conversion of the resulting CO to give an oxygen-containing compound by anaerobic fermentation in an aqueous medium, the fermentation also producing CO₂,
the partial oxidation step being carried out with the CO₂ produced during the fermentation.

### PRIORITY CLAIM

This application is a National Phase entry of PCT Application No. PCT/FR2011/052222, filed Sep. 26, 2011, which claims priority from FR Application No. 10 57872 filed Sep. 29, 2010, both of which are hereby incorporated by referenced herein in their entirety.

### FIELD OF THE INVENTION

The present invention relates to a process for producing an oxygen-containing compound, in particular ethanol, from light hydrocarbons, typically methane. A subject of the invention is also the cogeneration of electricity.

### BACKGROUND OF THE INVENTION

Processes for the fermentation of CO to give oxygen-containing compounds, in particular ethanol, are known. For example, the companies Coskata, Inc. (www.coskata.com) and LanzaTech (www.lanzatech.co.nz) have developed such processes, which use a mixture containing CO, generally synthesis gas. Anaerobic microorganisms have been developed, as have specific bioreactors, for carrying out the conversion of CO, or a feedstock containing CO, in particular into alcohols and carboxylic acids, such as ethanol, acetic acid, etc.

The feedstock of such processes is therefore a mixture containing CO. This feedstock can have various origins, it is most commonly synthesis gas or syngas, itself originating, for example, from the gasification of biomass, or other processes based on coal, or natural gas, such as SMR (Steam Methane Reforming) or ATR (AutoThermal Reforming) or else hydrocarbon PDX (Partial Oxidation).

All the current processes for preparing the feedstock for anaerobic fermentation have the common factor of containing both CO and hydrogen. This is in particular true of the processes described, for example, in WO 2009/064200 and WO 2008/028055, which use a bacterium of the Clostridium genus. A low H₂:CO ratio is sought for fermentation processes since the latter operate better, in terms of productivity and of microorganism growth stability, with a feedstock of pure CO or CO containing a small amount of hydrogen (see, for example, Mark E. Zappi, “Production of Ethanol from Synthesis Gas Using Fermentation,” Dave C. Swalm School of Chemical Engineering of the Mississippi State University, Jul. 28, 2003, http://www.che.msstate.edu/academics/faculty_staff/Zappi/consortium/production_of_ethanol_from_synth.htm). Nevertheless, the absence of hydrogen in these fermentation processes has the drawback of resulting in an undesirable emission of CO₂.

Anaerobic fermentation processes which operate efficiently while at the same time emitting a reduced amount of CO₂ compared with the prior art are therefore sought.

### SUMMARY OF THE INVENTION

The present invention includes implementing a process comprising a first step of synthesis of CO substantially free of hydrogen, by catalytic decomposition of a hydrocarbon-based feedstock, and then partial oxidation, the CO produced subsequently being used in an anaerobic fermentation process in which the CO₂ produced during the fermentation is recycled for the synthesis of CO.

Catalytic decomposition processes are in particular described in the publications by TAKENATA S. et al, Fuel, IPC Science and Technology Press, Guildford, G B, Vol. 83, No. 1, Jan. 1, 2004, pp. 47-57 and by AMMENDOLA P. et al., Combustion Science and Technology, Gordon and Breach Science Publishers, New York, US, Vol. 180, No. 5, Jan. 5, 2008, pp. 869-882. These processes comprise a step of partial oxidation for regenerating the catalyst. It is not envisaged to use the CO thus produced in a fermentation process.

The subject of the invention is a process for producing an oxygen-containing compound chosen from C₁ to C₄ alcohols or carboxylic acids, and mixtures thereof, comprising the following steps:
a step of catalytic decomposition of a hydrocarbon-based feedstock to give carbon, said step also producing hydrogen;
partial oxidation of the resulting carbon to give CO, using CO₂, the CO stream being separated from the hydrogen stream;
conversion of the resulting CO into an oxygen-containing compound by anaerobic fermentation in an aqueous medium, the fermentation also producing CO₂,
   the partial oxidation step being carried out with the CO₂ produced during the fermentation.

Embodiments of the invention are the following:
the oxygen-containing compound is chosen from ethanol, butanol, acetic acid and butyric acid, and mixtures thereof,
the process also comprises an additional step of separation of the oxygen-containing compound/water,
the hydrocarbon-based feedstock is methane or natural gas,
the process also comprises a step of recovery of the heat generated during the partial oxidation. The heat recovered can be used for the catalytic decomposition step and/or in another part of the process, in particular an additional step of separation of the oxygen-containing compound/water,
the catalytic decomposition and partial oxidation steps are integrated,
the catalytic decomposition and partial oxidation steps are carried out in a fixed, transported or fluidized bed,
the partial oxidation step is carried out with an oxidizing agent mixed with the CO₂ and chosen from oxygen, air, oxygen-enriched air, or a mixture thereof,
the hydrogen produced during the catalytic decomposition step can be used in a turbine or in a fuel cell,
the process is of use for the cogeneration of electricity.

The process according to the invention has the advantage of starting from a feedstock which is a feedstock substantially comprising pure CO. Such a feedstock is particularly suitable for the anaerobic fermentation processes that will be described below.

The process also offers the advantage of separating, from the beginning, the two streams, CO and H₂, so as to use each of them optimally. The hydrogen coproduced by the process according to the invention can be used in all processes that make use of hydrogen, in particular petroleum processes, for example the treatment of sulphur-containing feedstocks, hydrotreatments in general, hydrocracking, the production of aqueous ammonia, etc. One advantageous use of the hydrogen is the production of electricity, for example in a fuel cell or in a gas turbine suitable for the production of electricity. The CO produced is the feedstock for the fermenter.

The process according to the invention also offers the advantage of reducing CO₂ emissions.

### BRIEF DESCRIPTION OF THE FIGURES

FIG. 1 represents schematically a process according to the invention.

FIG. 2 represents the emission of CO₂ as a function of the electrical efficiency of a fuel cell.

### DETAILED DESCRIPTION OF THE FIGURES

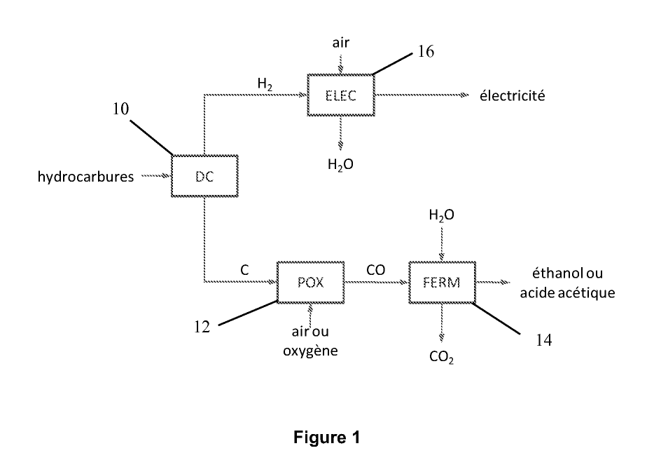

The invention is now described in greater detail and in a nonlimiting manner in the description which follows, with reference to FIG. 1, which describes a process according to the invention.

This process according to the invention comprises the following major steps. Firstly, the catalytic decomposition (CD) 10 of a hydrocarbon-based feedstock produces solid carbon C, which is partially oxidized 12 to give CO. These two steps represented independently in FIG. 1 are preferably integrated into a single step as described below. The CO is subsequently sent to a fermenter 14 for anaerobic fermentation in an aqueous medium. The fermentation produces, for example, ethanol or other oxygen-containing compounds, such as acetic acid. The fermentation process also produces CO₂. The (liquid) effluents resulting from the fermentation are subsequently separated in a conventional manner. The CD also produces a stream of hydrogen which can be used in particular for the production of electricity 16, in a fuel cell or in a turbine, water being generated subsequent to reaction with oxygen. The various steps are described in greater detail below.

### Catalytic Decomposition and Partial Oxidation

The first step is the step of catalytic decomposition, CD 10, which produces mainly two streams, one of carbon and the other of hydrogen, from hydrocarbons, in particular from methane, ethane, liquefied petroleum gases, light naphthas, etc. Methane or natural gas are the preferred feedstocks. Next, the carbon is partially oxidized so as to form CO.

During the first step, the methane (or another hydrocarbon) decomposes to give carbon and hydrogen, according to the following endothermic reaction:

CH₄→C+2H₂

The carbon will then accumulate on the catalyst, whereas the hydrogen (optionally with the residual feedstock) is withdrawn from the reactor. After a certain amount of time, the catalyst is completely covered and must be regenerated. The regeneration is preferably carried out by partial oxidation, so as to maximize the production of CO and to minimize the production of CO₂.

The carbon is oxidized according to the following reaction:

C+CO₂→2CO

When the regeneration gas also comprises oxygen (or air), the following parallel reactions then take place:

C+½O₂→CO

C+O₂→CO₂

The CO₂ produced by the regeneration reaction is also, in turn, used in the combustion reaction. The production of CO can be promoted by the operating conditions, in particular the temperature, and also a residual deposit of carbon on the catalyst.

The combination of the catalytic decomposition (CD) and the partial oxidation (PDX) 12 therefore produces two separate streams, composed essentially of hydrogen on one side and CO on the other. The first phase of the reaction (generation of solid carbon and of hydrogen) is endothermic and generally takes place at high temperature, for example 500 to 1500° C., advantageously 800 to 1300° C. The catalyst on which the reaction is performed is conventional; it may, for example, be Pd, Pt, Rh, Ru, Ir, Re, Co, Ni, W, Fe, Mn, Mo, Ta, Cu, Ag, Cr, Ti and V. Pd, Rh, Ru, Co, Ni and Fe are preferred. The catalyst is generally supported. A thermally stable inert support can be used as a mixture in order to increase the heat capacity. A catalyst based on silica-supported nickel can, for example, be used. The second phase of partial oxidation PDX 12 is exothermic, and produces slightly more heat than the first phase. It is possible to design a virtually autothermal reactor. It is also possible to provide internal and external heat exchangers for better thermal integration of the reactor. For example, during the production of hydrogen, the temperature of the catalyst can decrease by 200° C. (for example from 1000° C. to 800° C.), whereas the temperature will increase during the combustion phase. The excess calories produced during the oxidation will be recovered in order to use them elsewhere in the process (in particular at the separation level).

For details, reference may be made to the following publications: M. Matsukata, T. Matsushita, and K. Ueyama, “A novel hydrogen/syngas production process: catalytic activity and stability of Ni/SiO₂,” Chemical Engineering Science 51, no. 11 (1996): 2769-2774; and also M. G. Poirier and C. Sapundzhiev, “Catalytic decomposition of natural gas to hydrogen for fuel cell applications,” Int. J. Hydrogen Energy 22, no. 4 (1997): 429-433.

The two steps of production of H₂ and of CO are separated either in space (different reactors with circulation of catalyst between the two) or in time (static catalyst and alternating methane/oxidizing agent feed). Two independent streams of, on the one hand, substantially pure H₂ and of, on the other hand, substantially pure CO are obtained. It is therefore possible to use several reactors in parallel in alternation, which corresponds to continuous operation. A carousel can be provided in order to rotate the reactors. It is also possible to provide a reactor with a rotary inner part, the reaction zones being divided up into sections inside the reactor. Conventionally, the reactors are associated via a system of valves (manifolding). Given the various durations during the various steps, several reactors allocated to a particular step can be provided, with a shift of the inlets/outlets at the level of the various reactors.

Another solid, used for its heat capacity, can also be mixed with the solid catalyst. Such a thermal-ballast-forming solid may be silica, alumina, or else sand.

The solid (catalyst) is preferably fluidized by the gas in the reactor in order to advantageously benefit from the solid homogeneity properties in fluidized bed reactors. Although fluidized beds are preferred, fixed beds are also possible, as are transported beds. In the case of fluidized beds, the size and the particle distribution of the solid particles (whether particles of solid catalyst or mixtures with a thermal ballast) are chosen in a manner known to those skilled in the art so as to have a fluidized bed under the reaction conditions. By way of example, the dv50 of the catalyst particles is between 50 μm and 400 μm, preferably between 100 μm and 300 μm.

### Anaerobic Fermentation

The second step is a step of anaerobic fermentation 14, at the end of which oxygen-containing derivatives are produced.

This step is known and makes use of microorganisms, and a description of such processes can be found in, for example, Das, A. and L. G. Ljungdahl, Electron Transport System in Acetogens or Drake, H. L. and K. Kusel, Diverse Physiologic Potential of Acetogens; see also in particular Chapters 14 and 13 of Biochemistry and Physiology of Anaerobic Bacteria, L. G. Ljungdahl eds, Springer (2003).

Any microorganism capable of converting CO can be used. In particular, use is made of bacteria of the Clostridium genus, such as Clostridium carboxidivorans (for example for the production of ethanol and of n-butanol), Clostridium ragsdalei (for example for the production of ethanol), Butyribacterium methylotrophicum (for example for the production of ethanol and of butyric acid) or Clostridium ljungdahli (for example for the production of ethanol and of acetic acid).

The anaerobic conditions are those in particular under which the oxygen content is less than 1000 ppm, advantageously less than 100 ppm, in particular less than 10 ppm.

The temperature is generally ambient temperature, but any temperature suitable for the microorganism can be used. The pressure is generally atmospheric pressure.

In the synthesis of an oxygen-containing compound from natural gas, the microbiological route is advantageous compared with the route of an inorganic catalyst. Indeed, fermentation processes have many advantages: high selectivity; complete conversion possible, doing away with the equilibrium conversion imposed by the thermodynamics of gases; and tolerance to poisons present in the syngas.

Although the inorganic route for the synthesis of ethanol requires a precise syngas composition, with an H₂/CO ratio equal to 2, the biological route can accept very varied compositions. It is thus possible to convert CO all alone, the hydrogen required for the formation of ethanol originating from water. The invention is consequently advantageous since the fermentation process feedstock consists of substantially pure CO produced by the CD/PDX step described above.

The CO₂ produced during the fermentation is used for the partial oxidation of carbon in the preceding partial oxidation step. An external heat source can be provided if this proves to be necessary in order to re-use all of the CO₂.

The oxygen-containing compounds, such as ethanol and/or acetic acid, produced are generally separated from the water by a conventional liquid/liquid separation process (for example by distillation). This process is generally integrated into the fermentation unit. The separation requires a supply of energy, which can come from the heat released by the partial-oxidation or electricity-generating units, or else be supplied by an external source. The process according to the invention is advantageously integrated in terms of the supplies of heat at the various levels.

The invention therefore enables the production of liquid fuels such as ethanol.

### Production of Electricity

The stream of hydrogen gas which is produced can be used for many applications, such as the treatment of sulphur-containing feedstocks, hydrotreatments in general, hydrocracking, the production of aqueous ammonia, etc.

If required, the process according to the invention can also comprise a step of purification of the H₂, in particular by pressure swing adsorption.

One advantageous use of the hydrogen is the production of electricity 16, for example in a fuel cell or in a gas turbine suitable for the production of electricity. The electricity-producing unit requires a supply of air and generates water as main waste. It also releases heat which can be recovered and reused in other parts of the process.

The various technologies, such as those using a turbine or a fuel cell, are known to those skilled in the art and the latter may refer to the reference books on these subjects.

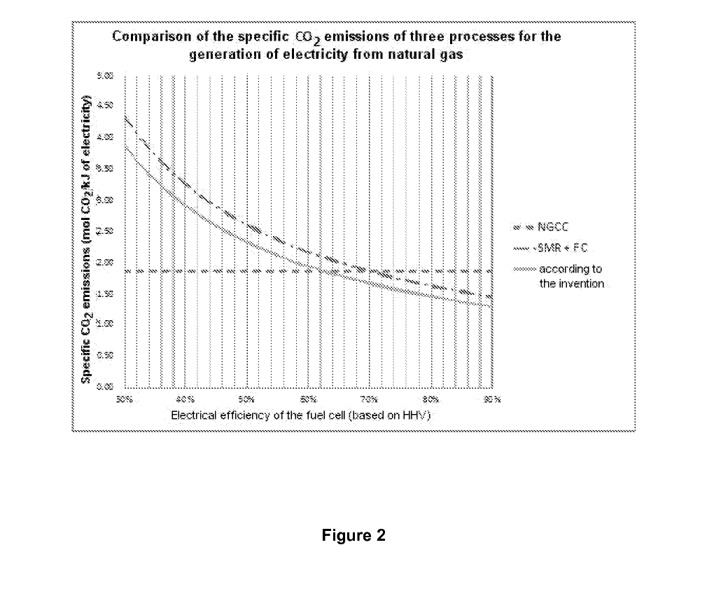

The invention offers many advantages compared with the known processes of the prior art, in particular SMR or NGCC (Natural Gas Combined Cycle, a technique known for the production of electricity).
The main advantage of the process according to the invention is that it clearly separates the hydrogen and the carbon contained in the raw material and therefore allows optimum use of each of these components. Hydrogen is the fuel of choice for the generation of electricity by means of fuel cells with a high energy yield. Carbon, on the other hand, is best used in the production of liquid fuels, to which it gives a high energy density, and in particular in fermentation processes which can be optimized for a feedstock which is substantially pure CO.
The result of this optimized use is a minimization of CO₂ emissions per unit of energy obtained. This will become apparent on reading FIG. 2, as detailed below.
The present process is equilibrated in terms of heat balance. Indeed, in the case of the production of an oxygen-containing derivative by fermentation, it is necessary to have a raw material containing CO. The conversion of natural gas (methane) to H₂ and CO involves either a release of heat (exothermic processes such as PDX) or a supply of external heat (endothermic processes such as SMR or CO₂ reforming). The process according to the invention uses a first endothermic step (generation of carbon) and a second step of regeneration by PDX which is exothermic with a slightly exothermic balance, which is optimal from a heat balance point of view. The processes according to the prior art are either strongly exothermic (PDX on methane) or strongly endothermic (SMR, CO₂ reforming) and are not optimized from a heat balance point of view.

FIG. 2 compares the CO₂ emissions per unit of electricity produced for three processes for generating electricity from natural gas: 1) natural gas combined cycle (NGCC), which represents the prior art; 2) steam methane reforming (SMR) followed by a fuel cell (SMR+FC); 3) process according to the invention.

The hypotheses used to construct this graph are the following. The CO₂ emissions per mole of H₂ are firstly taken into account, and then the amount of electricity per mole of H₂ is calculated. The CO₂ emission per kJ of electricity produced is thus obtained. The electrical efficiency is expressed relative to the higher heating value (HHV) which is the amount of energy given off by the complete combustion of one unit of fuel, the steam being assumed to be condensed and the heat recovered. The electrical efficiency of the fuel cell, expressed as % of the HHV, is thus obtained.

Thus, the conversion efficiency of the fuel cell on the basis of the HHV is given along the x-axis. The CO₂ emission per kJ of electricity produced is given along the y-axis.

The figure therefore comprises three curves, the first in fact being a straight line corresponding to the NGCC natural gas combined cycle. It is accepted that the electrical efficiency of the NGCC corresponds to approximately 60% of the HHV. Since there is no fuel cell, the curve for the NGCC is a straight line which simply takes into account the efficiency of the turbine. Given the current technology, the situation is close to the theoretical limit.

For SMR, an energy efficiency of 86% on the basis of the HHV is considered. This measurement takes into account the endothermic nature of the process.

In the case of the process according to the invention, the specific emission of CO₂ was calculated by dividing the emissions due to the process by the amount of electricity obtained. The ethanol (or another oxygen-containing compound) coproduced will ultimately generate, if it is burnt, additional CO₂ emissions. However, since this ethanol will displace in the market an amount of petrol that is equivalent in terms of energy content, it will at the same time avoid the CO₂ emissions due to the petrol. The calculation shows that the emissions thus avoided would be equal to or even slightly greater than those due to the combustion of the ethanol, therefore it is justifiable to exclude, in the calculation of the specific emissions of the process according to the invention, those due to the ethanol. Moreover, the conservative hypothesis was retained, stating that there is neither any external provision of energy in the process nor any export of energy to the exterior; in actual fact, the process according to the invention generates energy and therefore the curve should in actual fact be even lower than represented.

The energy efficiency of the fuel cell varies along the x-axis.

It will be noted that the curve for an SMR process intersects the straight line corresponding to the NGCC for an energy efficiency value of 70% for the fuel cell. This corresponds well to the NGCC efficiency, 86%×70%, namely at 60%.

It is noted that, in any event, the curve for the process according to the invention is lower than for an SMR process, and lower than the NGCC, the current reference, as long as the efficiency of the cell exceeds 62%, which is now commonly reached.

The embodiments above are intended to be illustrative and not limiting. Additional embodiments may be within the claims. Although the present invention has been described with reference to particular embodiments, workers skilled in the art will recognize that changes may be made in form and detail without departing from the spirit and scope of the invention.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

1. A process for producing an oxygen-containing compound chosen from C₁ to C₄ alcohols or carboxylic acids, and mixtures thereof, comprising the following steps:
a step of catalytic decomposition of a hydrocarbon-based feedstock to give carbon, said step also producing hydrogen;
partial oxidation of the resulting carbon to give CO, using CO₂, the CO stream being separated from the hydrogen stream;
conversion of the resulting CO into an oxygen-containing compound by anaerobic fermentation in an aqueous medium, the fermentation also producing CO₂,
the partial oxidation step being carried out with the CO₂ produced during the fermentation.

2. The process according to claim 1, in which the oxygen-containing compound is chosen from ethanol, butanol, acetic acid and butyric acid, and mixtures thereof.

3. The process according to claim 1, also comprising an additional step of separation of the oxygen-containing compound/water.

4. The process according to claim 1, in which the hydrocarbon-based feedstock is methane or natural gas.

5. The process according to claim 1, comprising a step of recovery of the heat generated during the partial oxidation.

6. The process according to claim 5, in which the heat recovered is used for the catalytic decomposition step and/or in another part of the process, in particular an additional step of separation of the oxygen-containing compound/water.

7. The process according to claim 1, in which the catalytic decomposition and partial oxidation steps are integrated.

8. The process according to claim 1, in which the catalytic decomposition and partial oxidation steps are carried out in a fixed, transported or fluidized bed.

9. The process according to claim 1, in which the partial oxidation step is carried out with an oxidizing agent which is CO₂ mixed with oxygen, air, oxygen-enriched air, or a mixture thereof.

10. The process according to claim 1, in which the hydrogen stream is used in a turbine or in a fuel cell.

11. The process according to claim 1, for the cogeneration of electricity.
